(19) United States
(12) Patent Application Publication
(10) Pub. No.: US 2010/0144008 A1
(43) Pub. Date: Jun. 10, 2010

(54) TREATMENT OF FABRY DISEASE

(75) Inventor: Johannes Maria Franciscus Gerardus Aerts, Abcoude (NL)

Correspondence Address: YOUNG & THOMPSON, 209 Madison Street, Suite 500, Alexandria, VA, 22314, US

(73) Assignee: ACADEMISCH ZIEKENHUIS BIJ DE UNIVERSITEIT VAN AMSTERDAM, Amsterdam (NL)

(21) Appl. No.: 12/524,209

(22) PCT Filed: Dec. 21, 2007
(86) PCT No.: PCT/NL2007/050684

§ 371 Date: Jan. 19, 2010

Related U.S. Application Data
(60) Provisional application No. 60/871,334, filed on Dec. 21, 2006.

(51) Int. Cl. C12N 9/00

(52) U.S. Cl. 435/183

(57) ABSTRACT

A pathogenic factor in plasma as an improved therapy for Fabry disease.

### FIELD OF THE INVENTION

The present invention is in the field of Fabry disease and concerns an improvement in the treatment of Fabry disease.

### BACKGROUND OF THE INVENTION

Fabry disease is one of several genetically inherited diseases called lysosomal storage disorders. It causes a wide range of signs and symptoms that can range from mild to severe and life threatening. Fabry disease, also known as angiokeratoma corporis diffusum universale, Morbus Fabry, and Anderson-Fabry disease, is a progressive, X-chromosome-linked genetic disorder resulting from a defect in the gene for the lysosomal enzyme alpha-galactosidase A (alpha-GAL). This enzyme deficiency results in an accumulation of glycosphingolipids, particularly globotriaosylceramide (also abbreviated as Gb3, GL-3, or ceramide trihexosamide (CTH)), in the vascular endothelium and visceral tissues throughout the body. Because clinical presentation is widely variable and symptoms may mimic those of other diseases, diagnosis of Fabry disease is often overlooked or delayed. Despite being an X-linked disorder, some females may express varying degrees of clinical manifestations. Moreover, there are variants of Fabry disease that do not present with classical signs and symptoms. This suggests that the actual incidence of Fabry disease may be higher than currently estimated incidence of 1 in 40,000 males (panethnic).

Since Fabry is X-linked, the disease predominantly affects males (hemizygotes), who have little if any endogenous alpha-GAL. Although X-linked recessive diseases generally do not affect females, there are female carriers (heterozygotes) who may experience varying degrees of disease manifestations. It is believed that X-chromosomal inactivation (lyonization), which can block expression of the functional alpha-GAL gene in all or some parts of the body, is responsible for disease onset in carriers. Although the prevalence of female carriers who develop overt clinical manifestations is unknown, recent studies indicate that manifestations in carrier females are more common than previously thought.

The inability to catabolize Gb-3 leads to progressive multisystemic damage to the kidney, heart, and cerebrovascular system. The clinical course of Fabry disease is usually marked by chronic pain, angiokeratomas, hypohidrosis, heat and cold intolerance, corneal opacities, renal failure, stroke, and cardiac complications. As the disease progresses, complications may become life-threatening. Progressive organ and tissue damage associated with Fabry disease may result in substantially decreased life expectancy. Before the availability of renal dialysis or transplantation, the average age of death among patients with classical Fabry disease was 41 years; today, average life expectancy is still only 50 years.

Signs and symptoms associated with Fabry disease are widely varied, making diagnosis challenging. Clinical onset usually occurs in childhood or adolescence, but symptoms are frequently misinterpreted or overlooked. Accurate diagnosis is frequently not established until adulthood, when the disease has progressed, and organ dysfunction or failure has occurred.

The cardinal presenting features of Fabry disease are intermittent acroparesthesia and episodic crises of pain and fever (especially in childhood), angiokeratomas, hypohidrosis, heat and cold intolerance, and a characteristic “whorled” corneal opacity that does not affect vision. Progressive accumulation of Gb3 in the vascular endothelium and other tissues leads to life-threatening manifestations in adulthood involving the heart, kidneys, central and peripheral nervous system, and cerebrovascular system.

Below an overview is provided of the signs and symptoms of Fabry disease that may be seen at different stages of life. Both male and female patients may experience some or all of these manifestations to varying degrees, depending in part on the extent of alpha-GAL activity levels.

Childhood: episodic pain crises, acroparesthesia; hypohidrosis; corneal and lenticular opacities; recurrent fever; heat and cold intolerance.

Adolescence: gastrointestinal manifestations; angiokeratomas; fatigue; episodic pain crises, acroparesthesia; hypohidrosis; corneal and lenticular opacities; recurrent fever; heat and cold intolerance.

Adulthood: renal insufficiency/failure; neurological complications; cerebrovascular disease; cardiac dysfunction; hearing loss and tinnitus; gastrointestinal manifestations; angiokeratomas; fatigue; episodic pain crises, acroparesthesia; hypohidrosis; corneal and lenticular opacities; recurrent fever; heat and cold intolerance.

Growing evidence indicates there may be a significant number of “atypical variants”—hemizygotes who have few or none of the hallmark symptoms of classical Fabry disease. Atypical variants have residual plasma alpha-GAL levels (1% to 30% of normal) and present much later in life than patients with classical Fabry disease. They are often identified serendipitously, and usually have manifestations predominately in one organ system.

Clinical heterogeneity and the rarity of Fabry disease makes diagnosing Fabry disease a challenge. The age of presentation, presenting symptoms, and clinical course vary from individual to individual. Greater recognition of Fabry disease symptoms may lead to earlier suspicion and diagnosis, which in turn may result in more effective disease management.

Any of the symptoms described above may lead to a presumptive clinical diagnosis of Fabry disease. However, because Fabry disease is a multisystemic disorder, patients may present different symptoms to a wide range of specialists. One confounding factor in diagnosis is the fact that many common signs and symptoms of Fabry disease are misattributable to other conditions.

Once a presumptive diagnosis of Fabry disease has been made based on clinical signs and symptoms, definitive diagnosis can be made by testing for deficient alpha-gal enzyme activity in plasma, leukocytes, tears, or biopsied tissue.

Although females carrying the Fabry gene may be asymptomatic or present with mild clinical manifestations, definitive identification of carriers is important. Diagnosis allows practitioners to monitor for new or worsening symptoms, and can help with identifying other family members with the disease.

Affected females can be diagnosed with Fabry disease by very low or absent alpha-GAL activity and by lipid deposition in biopsied tissues or urinary sediment. Many female carriers (with or without symptoms) have below-normal levels of alpha-GAL activity and/or the characteristic corneal opacities. However, this is not true for all carriers—some have alpha-GAL activity in the low to normal range. In families with an identified mutation, mutation analysis is the definitive way to identify carrier females. In families for whom a specific mutation is not documented, linkage analysis can be performed to establish carrier status.

Disease management strategies may include medications and lifestyle approaches to symptom relief and interventions to delay serious sequelae due to organ damage (eg, kidney transplantation, cardiac pacemaker insertion).

While symptom management may improve a patient's quality of life, treatment to prevent or reverse accumulation of Gb3 and offers the potential to stem disease progression and prevent organ damage.

Gene therapy for Fabry disease is in the early stages of investigation. Research has identified a couple of different approaches that show potential promise in pre-clinical studies in Fabry mice. Research has also identified two approaches involving “small molecules.” Both of these require some residual alpha-GAL activity to be effective and could potentially be used in conjunction with either gene therapy or enzyme replacement therapy. The first approach involves substrate inhibition therapy to reduce cellular synthesis of glycosphingolipids. Two potentially promising small molecules are N-butyldeoxynojirimycin and D-threo-1-ethylendioxyphenyl -2-palmitoylamino-3-pyrrolidino-propanol (D-t-EtDO-P4). A second approach involves use of a competitive inhibitor, in particular 1-deoxy-galactonojirinmycin, of alpha-GAL to increase the activity of residual enzyme.

Enzyme replacement therapy (ERT) is currently available in the United States and in over 27 additional countries for people with Fabry disease. Following the success of enzyme replacement therapy for type 1 Gaucher disease, comparable therapies have been developed for the treatment of Fabry disease. Chronic intravenous administration of the registered recombinant alpha-galactosidase preparations (agalasidase alpha (Replagal®, Shire) and agalsidase beta (Fabrazyme®, Genzyme) aims to correct the alpha-galactosidase A deficiency in cells of Fabry patients and thus to reverse, or at least stop, storage material accumulation in lysosomes and the accompanying pathological processes. Unfortunately it has become clear that clinical responses to enzyme replacement therapies in Fabry patients are far less spectacular than those shown by Gaucher patients receiving a comparable intervention.

To improve the efficacy of enzyme replacement therapies for Fabry disease additional insight is clearly needed regarding the impact of the common formation of (neutralizing) antibodies directed against the therapeutic enzymes in male Fabry patients. Needed is better insight regarding optimal enzyme dosing regimens for individual Fabry patients. Furthermore, the most appropriate time of therapeutic intervention has to be established since it is found that the clinical impact of therapeutic intervention is considerably poorer in patients with already established extensive disease.

### DESCRIPTION OF THE INVENTION

It is so far believed that the pathogenesis of Fabry disease simply mimics that of other lysosomal disorders, for example type 1 Gaucher disease. The envisioned sequence of events is thus as follows. Alpha-galactosidase A deficiency causes accumulation of its corresponding substrate (the globoside Gb3). The lysosomal storage somehow results in cellular dysfunction and damage. This ongoing process finally leads to organ failures manifesting as clinical symptoms. There are several compelling indications that in reality the pathogenesis of Fabry disease is far more complex. Firstly, a considerable number of female Fabry heterozygotes develop a severe course of disease, closely resembling that of male Fabry hemizygotes completely lacking alpha-galactosidase A. The presence of alpha-galactosidase A competent cells and circulating enzyme in female heterozygotes apparently hardly prevents disease onset and progression. This phenomenon differs markedly from the situation in Hunter disease, another X-linked lysosomal storage disorder. Secondly, although most male Fabry hemizygotes completely lack alpha-galactosidase A, disease manifestation occurs nevertheless relatively late in life. Again this markedly differs from any other lysosomal storage disorder for which complete lack of degradation capacity is either incompatible with life or causes infantile phenotypes. Thirdly, genetically engineered Fabry mice quickly develop pronounced Gb3 storage in the endothelium but not the characteristic organ failures of Fabry patients. Apparently there is not a very close relation between primary Gb3 accumulation and pathogenic processes. It has to be concluded from the discussion above that some crucial link is missing in our present understanding of the pathogenesis of Fabry disease.

It is an object of the present invention to trace the crucial missing link in the pathogenesis of Fabry disease, in particular it is an object to find a way to improve the diagnosis of Fabry disease, which in turn allows the identification of improvements in the therapy of Fabry disease.

Upon extensively researching lipid profiles in Fabry patients, the present inventor has surprisingly found that that lyso-ceramide trihexosamide (lyso-CTH) is dramatically elevated in plasma of Fabry patients. Lyso-CTH is formed as side-product from ceramide trihexosamide (CTH), either by ceramidase or protease activity. Lyso-CTH is a potent inhibitor of both alpha-galactosidase A and B. Up to now there are no parameters that predict pathogenesis of Fabry disease and response to therapies. With the finding of the aberrant plasma-levels of lyso-CTH in Fabry patients a unique diagnostic tool for Fabry disease is provided. Thus, monitoring of lyso-CTH will offer a completely new tool that can actively guide clinicians in clinical decision making regarding start of (preventive) treatment and individualized dosing regimens for Fabry disease.

With this finding also an explanation of how Fabry disease generally can remain sub-clinical for almost one decade, where after the pathophysiological processes seem to accelerate in the third decade in male and the fourth decade in females. Firstly alpha-galactosidase B can partly eliminate alpha-galactosidase A deficiency. In other words, alpha-galactosidase B displays the same activity as alpha-galactosidase A towards Gb3, be it with a much lower Km towards the substrate. However, the gradual generation and release of a side product of the Gb3 storage material can inhibit alpha-galactosidase A and B. In deed it was confirmed that lyso-CTH inhibits both alpha-galactosidase A and B. This explains the late manifestation of Fabry disease and the comparable course of the disorder in males (virtually) lacking alpha-galactosidase A and females with competent cells and circulating alpha-galactosidase A protein.

With this finding, lyso-CTH itself also is identified as toxic component in Fabry disease. Lyso-CTH was found to promote in vitro smooth muscle cell proliferation at concentrations as occurring in plasma of Fabry patients. Smooth muscle cell proliferation leads to vascular aberrations and cardiac hypertrophy.

Based on the identification of lyso-CTH as a pathogenic factor in Fabry disease the following sequence of events in Fabry disease is postulated:
1 primary CTH storage (very early in males)
2 slow formation of lyso-CTH
3 lyso-CTH starts to inhibit alpha-galactosidase A and B
4 lyso-CTH exerts its deleterious effects

This finding now results in the notion that in order for a therapy to be effective in Fabry patients it is highly advantageous to degrade lyso-CTH in circulation, i.c. in plasma, rather than to hydrolyse CTH inside cells.

Thus the present invention concerns a method for the treatment of Fabry disease, said method comprising administering to a patient in need thereof a therapeutically effective amount of an agent that decreases plasma concentration of lyso-CTH.

In other words, the present invention concerns the use of an agent that decreases plasma concentration of lyso-CTH for the manufacture of a medicament for the treatment of Fabry disease.

The invention can also be worded as an agent that decreases plasma concentration of lyso-CTH for the treatment of Fabry disease.

Plasma concentration of lyso-CTH can be effectively reduced by hydrolyzing lyso-CTH. Thus, in one embodiment, the agent that decreases plasma concentration of lyso-CTH is a lyso-CTH hydrolyzing enzyme. Advantageously a lyso-CTH hydrolyzing enzyme hydrolyses lyso-CTH in plasma.

In order to hydrolyze lyso-CTH effectively in plasma it is beneficial to take measures that keeps the lyso-CTH hydrolyzing as long as possible in plasma. Thus, in one embodiment agent that decreases plasma concentration of lyso-CTH is a lyso-CTH hydrolyzing enzyme that is provided with means to sustain in circulation. In one embodiment the measure that is taken to keep lyso-CTH hydrolyzing enzyme as long as possible in plasma is to pegylate, i.e. attach polyethylenenglycol to, the lyso-CTH hydrolyzing enzyme.

A possibility is to optimize lyso-CTH hydrolyzing enzymes for hydrolysis in plasma. Thus in one embodiment the agent that decreases plasma concentration of lyso-CTH is a lyso-CTH hydrolyzing enzyme that is optimized for hydrolyzing lyso-CTH in plasma. Also a possibility is to optimize the optimum pH of a lyso -CTH hydrolyzing enzyme so that hydrolysis of lyso-CTH in plasma effectively takes place. In one embodiment the agent that decreases plasma concentration of lyso-CTH is a lyso-CTH hydrolyzing enzyme that has an optimum of hydrolyzing activity at pH 6.5-7.5.

As a starting point for optimization agalsidase alpha (Replagal®, Shire) and agalsidase beta (Fabrazyme®, Genzyme) can be taken and these enzymes can be pegylated or modified for example by site directed mutagenesis to optimize the pH optimum of the enzymes or to optimize the hydrolytic activity in plasma. Thus in one embodiment the present invention relates to a method for the treatment of Fabry disease, said method comprising administering to a patient in need thereof a therapeutically effective amount of modified agalsidase alpha or modified agalsidase beta wherein said modification results in improved hydrolysis of lyso-CTH in plasma compared to unmodified agalsidase alpha or unmodified agalsidase beta.

In other words, the invention concerns the use of modified agalsidase alpha or modified agalsidase beta wherein said modification results in improved hydrolysis of lyso-CTH in plasma compared to unmodified agalsidase alpha or unmodified agalsidase beta for the manufacture of a medicament for the treatment of Fabry disease.

Methods of measuring the concentration of lyso-CTH in plasma samples are available and known to those skilled in the art. For example measuring the concentration of lyso-CTH in plasma samples involves Bligh and Dyer extraction preferably followed by butanol/water extraction. Next the extracted lysosphingolipids, including lyso-CTH, may be derivatised with a label in order to facilitate detection. Analysis can be routinely carried out on a HPLC system preferably equipped with a reversed phase column. Preferably an internal standard is included in order to properly quantify the results obtained, i.c. measure the actual concentration of lyso-CTH in the plasma sample of the subject.

Alternatively HPLC-tandem MS can be used to analysis lyso-CTH in plasma samples. For example the simultaneous quantitative analysis of bioactive sphingolipids by high-performance liquid chromatography-tandem mass spectrometry is described by Bielawski et al in Methods. 2006 June; 39(2):82-91 which is incorporated herein by reference. In general there has been a recent explosion in research concerning novel bioactive sphingolipids (SPLs) such as ceramide (Cer), sphingosine (Sph) and sphingosine 1-phosphate (Sph-1P) that necessitates development of accurate and user-friendly methodology for analyzing and quantitating the endogenous levels of these molecules. ESI/MS/MS methodology provides a universal tool used for detecting and monitoring changes in SPL levels and composition from biological materials. Simultaneous ESI/MS/MS analysis of sphingoid bases (SBs), sphingoid base 1-phosphates (SB-1Ps), Cers and sphingomyelins (SMs) is performed on a Thermo Finnigan TSQ 7000 triple quadrupole mass spectrometer operating in a multiple reaction monitoring (MRM) positive ionization mode. Biological materials (cells, tissues or physiological fluids) are fortified with internal standards (ISs), extracted into a one-phase neutral organic solvent system, and analyzed by a Surveyor/TSQ 7000 LC/MS system. Qualitative analysis of SPLs is performed by a Parent Ion scan of a common fragment ion characteristic for a particular class of SPLs. Quantitative analysis is based on calibration curves generated by spiking an artificial matrix with known amounts of target synthetic standards and an equal amount of IS. The calibration curves are constructed by plotting the peak area ratios of analyte to the respective IS against concentration using a linear regression model. This robust analytical procedure can determine the composition of endogenous sphingolipids (ESPLs) in varied biological materials and achieve a detection limit at 1 pmol or lower level. This and related methodology are already defining unexpected specialization and specificity in the metabolism and function of distinct subspecies of individual bioactive SPLs.

Also the present invention relates to a method of monitoring and or a method of optimizing a therapy for Fabry disease, said method comprising measuring the concentration of lyso-ceramide trihexosamide (lyso-CTH) in a plasma sample of a subject. Preferably in these methods the concentration that is measured is compared with a standard concentration. A standard concentration of lyso-CTH is the average concentration of lyso-CTH in plasma samples of individuals that are known to have no deficiency of lysosomal enzyme alpha-galactosidase A. Preferably the standard concentration is measured for males and females separately as described above. Alternatively in these methods the concentration is measured after a therapeutic intervention and preferably is compared with a standard concentration as described above or is compared with the concentration of plasma lyso-CTH in the same subject prior to the therapeutic intervention. Optimisation of dosage, dosage form, route of administration, nature of therapeutic agent etc can be achieved in this way.

### EXAMPLES

Analysis

The formula below represent the structures of Gb3 (CTH) and lyso-CTH (lyso-Gb3)

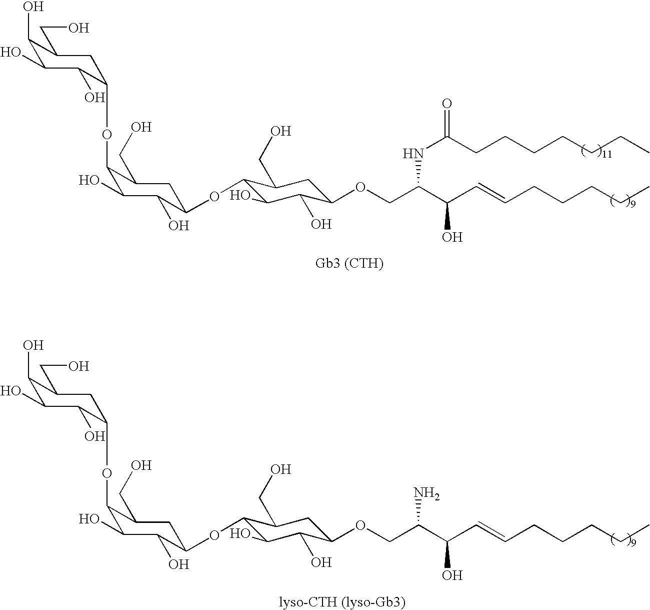

An optimal extraction procedure for lyso-CTH from plasma samples was established. A double extraction was carried out, first a Bligh and Dyer extraction followed by butanol extraction.

Partitioning of lyso-CTH (% of total)

| | upper phase | lower phase |
|---|---|---|
| Bligh and Dyer | 90 | 10 |
| butanol/water | 99 | 2 |

The concentration of lyso-CTH was measured as follows:

Plasma samples were extracted by the procedure of Bligh and Dyer. The upper phase was dried under N2 and subjected to butanol/water extraction. The upper phase was dried under N₂ and the residue was taken up in 250 μl methanol.

The residue, including lysosphingolipids, dissolved in methanol were derivatised on line for 30 min with o-phtalaldehyde. Analysis was performed using an HPLC system (Waters Associates, Milford, Mass.) and a Hypersil BDS C18 3μ, 150×4.6 mm reverse phase column (Alltech). Chromatographic profiles were analysed using Waters Millenium software. The eluent used was methanol:water; 88:12 (w/w).

By standard addition of lyso-CTH to normal plasma a calibration curve was constructed.

The tables below show the results of a number of samples that were analysed.

TABLE 1
Lyso-CTH in plasma of male members of family H

| Age | Genetic status | lyso-CTH (pmol/ml) |
|---|---|---|
| 50 | normal | 2 |
| 24 | normal | 3 |
| 20 | hemizygote | 253 |
| 18 | hemizygote | 166 |
| 16 | hemizygote | 315 |
| 11 | hemizygote | 483 |
| 9 | normal | 0 |

Thus in male Fabry patients, or males with a predisposition to become Fabry patients, a dramatically increase of plasma lyso-CTH is found. Plasma lyso-CTH in all tested symptomatic males >50 fold normal mean.

TABLE 2
Lyso-CTH in plasma of female members of family H

| Age | Genetic status | lyso-CTH (pmol/ml) |
|---|---|---|
| 45 | heterozygote | 83 |
| 42 | heterozygote | 19 |
| 17 | normal | 0 |
| 15 | heterozygote | 21 |
| 8 | normal | 0 |
| 7 | heterozygote | 0 |
| 5 | normal | 0 |

Thus starting form at least adolescence in female Fabry patients, or females with a predisposition to become Fabry patients, a dramatically increase of plasma lyso-CTH is found with concentration in the pmol/ml range whereas normal mean values are sub pmol/ml.

Toxicity

The toxic effect of lyso-CTH was determined in vitro in SMC-41 cell line. ³H thymidine incorporation (per 24 h) was determined as a function of lyso-CTH concentration. At concentrations of lyso-CTH as found in plasma of Fabry patients, i.e. 0.065-0.65 μM, increased isotope incorporation and increased proliferation of SMC-41 was found

Derivatization, Circulation and Activity of alphaGAL Replacement Enzyme

Via standard methods agalsidase beta (Fabrazyme®, Genzyme) is pegylated.

In a mouse model of Fabry disease at different time-points plasma samples are analysed for the presence of agalsidase beta to give reference values and at the same different time-points plasma samples are analysed for the presence of pegylated agalsidase beta. The results show an increase in plasma circulation time of pegylated agalsidase beta compared to underivatised agalsidase beta. Moreover simultaneous measurement of the concentration of lyso-CTH in the plasma samples reveals that the concentration of lyso-CTH is significantly lower in the samples of the animals receiving pegylated agalsidase beta compared to underivatised agalsidase beta.

1-8. (canceled)

9. A medicament for treatment of fabry disease, comprising an agent that decreases plasma concentration of lyso-CTH.

10. The medicament according to claim 9 wherein the agent that decreases plasma concentration of lyso-CTH is a lyso-CTH hydrolyzing enzyme.

11. The medicament according to claim 9 wherein the agent that decreases plasma concentration of lyso-CTH is a lyso-CTH hydrolyzing enzyme that hydrolyses lyso-CTH in plasma.

12. The medicament according to claim 9, wherein the agent that decreases plasma concentration of lyso-CTH is a lyso-CTH hydrolyzing enzyme that is provided with means to sustain in circulation.

13. The medicament according to claim 9, wherein the agent that decreases plasma concentration of lyso-CTH is a lyso-CTH hydrolyzing enzyme that is pegylated.

14. The medicament according to claim 9, wherein the agent that decreases plasma concentration of lyso-CTH is a lyso-CTH hydrolyzing enzyme that has an optimum of hydrolyzing activity at pH 6.5-7.5.

15. The medicament according to claim 9, wherein the agent that decreases plasma concentration of lyso-CTH is a lyso-CTH hydrolyzing enzyme that is optimized for hydrolyzing lyso-CTH in plasma.

16. The medicament according to claim 9, wherein the agent that decreases plasma concentration of lyso-CTH is modified agalsidase alpha or modified agalsidase beta wherein said modification results in improved hydrolysis of lyso-CTH in plasma compared to unmodified agalsidase alpha or unmodified agalsidase beta.
